(19) United States
(12) Patent Application Publication
(10) Pub. No.: US 2017/0176367 A1
(43) Pub. Date: Jun. 22, 2017

(54) APPARATUS TO MEASURE CONDUCTIVITY OF NON-AQUEOUS LIQUIDS AT VARIABLE TEMPERATURES AND APPLIED VOLTAGES

(71) Applicant: SHELL OIL COMPANY, Houston, TX (US)

(72) Inventors: Rodney Truman PENNINGTON, Houston, TX (US); Robert Paul ADAMSKI, Missouri City, TX (US); Gautam Chandrakanth KINI, Houston, TX (US)

(21) Appl. No.: 15/382,860

(22) Filed: Dec. 19, 2016

Related U.S. Application Data
(60) Provisional application No. 62/270,630, filed on Dec. 22, 2015.

(51) Int. Cl. G01N 27/06; G01N 1/42; G01N 33/28

(52) U.S. Cl. CPC G01N 27/06; G01N 33/2823; G01N 1/42

(57) ABSTRACT

A conductivity meter for measuring the conductivity of a sample of non-aqueous liquid. The conductivity meter includes a conductivity cell which contains the sample and which is disposed within a heater block which can control the temperature of the sample and a pressure vessel which can control the surrounding pressure for the conductivity cell. A probe senses conductivity of the sample as a voltage is applied across the sample.

### FIELD OF THE INVENTION

The invention relates generally to methods for measuring the conductivity of non-aqueous liquids, such as crude oils and crude oil blends.

### DESCRIPTION OF THE RELATED ART

The conductivity of non-aqueous liquids, such as crude oils is an important factor for determining how such liquids must be handled, stored and processed. If, for example, a certain liquid has high conductivity at a particular temperature, it would be important to store that liquid in a manner wherein it is protected from potential electrical discharge. Currently, there are difficulties measuring conductivity of non-aqueous liquids in a safe and effective manner. For example, samples can evaporate at the temperatures used during testing. Furthermore, at specific temperatures, the conductivity of crude oil can be grossly miscalculated due to polarization effects, which are typically seen upon application of a DC (direct current) field.

### SUMMARY OF THE INVENTION

The invention provides methods for effectively measuring conductivity of non-aqueous liquid samples or sample blends. In particularly preferred applications, the methods are useful for determining of the conductivity of samples of crude oil, blends of crude oil or other non-aqueous liquids at process temperatures and pressures.

An exemplary method of measurement is described wherein a sample is placed within the testing vessel of a conductivity cell. First and second electrodes are operably associated with the conductivity cell so that a voltage source can apply a voltage across the sample. The conductivity cell is preferably disposed within a heater block which is associated with a heating controller. The heating controller controls the temperature of the heating block and conductivity cell. Preferably, the heating controller is a cascade feedback controller which optimally adjusts temperature in a manner which will attain a desired temperature in a rapid fashion.

In accordance with a preferred method of measurement, the sample is pressurized using a pressure control system which subjects the sample to a pressure blanket of inert gas. Also in preferred embodiments, the pressure blanket is at a pressure that is significantly greater than the vapor pressure of the liquid sample.

After the liquid sample has been brought to a desired pressure and heated to a desired temperature, a voltage or series of voltages is/are applied across the first and second electrodes of the conductivity cell by the voltage source. The power source is preferably operable to apply varying levels of voltage to the sample for certain lengths of time. In particular embodiments, DC voltage is applied in sequences of varying levels over short intervals of time, such as 1 second or less. The RTD immersed within the sample detects the current induced through the liquid sample by the application of voltage.

In accordance with an exemplary method of determining conductivity of a liquid sample, a liquid sample is placed within the testing vessel and an RTD (resistance temperature detector) is immersed into the liquid sample, thereby forming a conductivity cell. The conductivity cell is retained within a heater block and further connected to the pressure control system. The conductivity cell is maintained under pressure using an inert gas pressure blanket, after which the heater block is heated to a desired temperature by the heating controller. A programmable data acquisition system is used to specify the temperatures at which voltage needs to be applied, the type of voltage (DC or AC) that needs to be applied, the desired time duration for which voltage should be applied and the sequence of voltages (usually ranging from 1V-100V) which need to be applied. When the desired voltage is applied to the liquid sample in the annular region of the conductivity cell, a conductivity probe measures the current within the sample as voltage is applied across it. During testing, the temperature of and pressure applied to the liquid sample may be varied, which allows measurement of conductivity in a variety of conditions. In accordance with some embodiments, the voltage (type, magnitude and time duration of application) which is applied to the liquid sample can be adjusted during testing.

Measuring conductivity of crude oil is non-trivial, especially at elevated temperatures where lighter blends of the crude oil tend to evaporate and in turn change the composition (and resultant conductivity) of the crude oil blend. The evaporation potential is reduced by application of a pressure blanket that is maintained at a pressure that is sufficiently higher than the vapor pressure of the liquid sample to prevent any of the sample from escaping via evaporation. A pressure transducer attached to the conductivity cell and the data acquisition system helps monitor the pressure of the sample.

### BRIEF DESCRIPTION OF THE DRAWINGS

For a thorough understanding of the present invention, reference is made to the following detailed description of the preferred embodiments, taken in conjunction with the accompanying drawings, wherein like reference numerals designate like or similar elements throughout the several figures of the drawings and wherein:

FIG. 1 is a side, cross-sectional view of an exemplary testing vessel portion of a conductivity cell constructed in accordance with the present invention.

FIG. 2 is a side, exterior view of the testing vessel shown in FIG. 1, now turned 90 degrees.

FIG. 3 is a top view of the testing vessel.

FIG. 4 is side, cross-sectional view of an exemplary conductivity cell containing a liquid sample whose conductivity is to be tested.

FIG. 5 is a side view of an exemplary heater block containing the conductivity cell shown in FIG. 1-3.

FIG. 6 depicts a conductivity meter wherein the conductivity cell and heater block are contained within an outer pressure vessel.

FIG. 7 is a diagram depicting steps of an exemplary method for measuring conductivity of a sample of non-aqueous liquid.

### DETAILED DESCRIPTION OF THE PREFERRED EMBODIMENTS

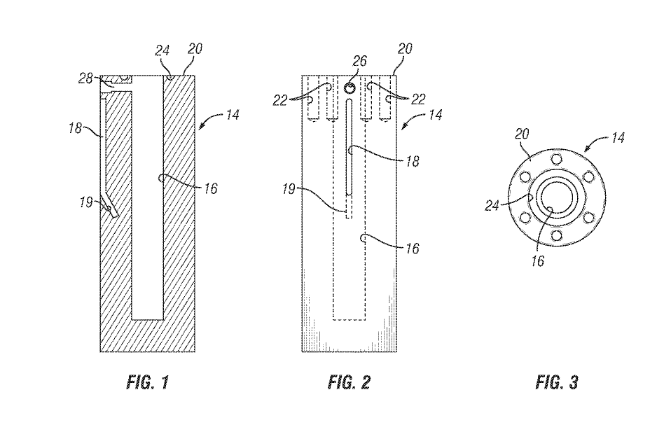

FIGS. 1-6 depict features of an exemplary conductivity meter 10 constructed in accordance with the present invention. The conductivity meter 10 includes a conductivity cell 12, portions of which are illustrated in FIGS. 1-4. FIGS. 1-3 depict a cylindrical testing vessel 14 which defines a liquid sample chamber 16 within. An electrode cavity 18 is formed on the exterior radial surface of the testing vessel 14. The cross-sectional view of FIG. 1 shows that the electrode cavity 18 preferably has an angled lower portion 19 which extends radially inwardly into the body of the testing vessel 14. As FIGS. 2 and 3 depict, the upper surface 20 of the testing vessel 14 preferably includes threaded openings 22 and an O-ring groove 24. A lateral port 26 is disposed through the testing vessel 14 and permits a liquid sample within the testing vessel 14 to be exposed to external pressure.

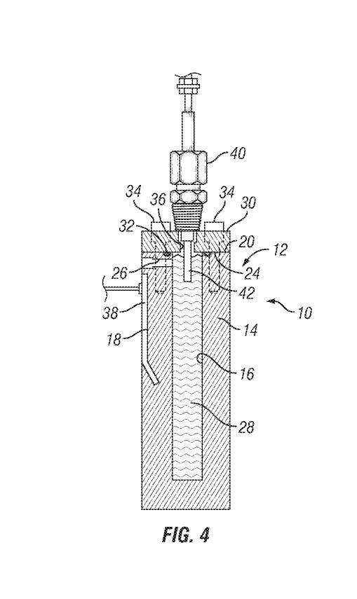

FIG. 4 depicts the conductivity cell 12 assembled with a non-aqueous liquid sample 28 within the liquid sample chamber 16 and a cap 30 which has been secured to the upper surface 20 of the testing vessel 14. O-ring 32 is seated in O-ring groove 24 and provides a fluid seal between the testing vessel 14 and cap 30. Threaded connectors 34 secure the cap 30 to the testing vessel 14. The cap 30 has a central opening 36 formed therein. It is noted that the testing vessel 14 and cap 30 are preferably formed of stainless steel. A first conductive electrode 38 is shown seated within the electrode cavity 18. In accordance with preferred embodiments, the non-aqueous liquid making up the sample 28 is crude oil or another hydrocarbon liquid. Also, it is noted that use of the term “non-aqueous” in this description does not require strict absence of water. Rather, “non-aqueous” liquids, as referred to herein, will refer to liquids which contain either no water or small percentages of water which do not appreciably affect the conductivity of the liquid.

A resistance temperature detector (RTD) 40 is inserted through the central opening 36 and into the liquid sample 28. The resistance temperature detector 40 presents a conductive distal end 42 which extends into the liquid sample 28 and is operable to function as a second electrode for the conductivity cell 12. In addition, the resistance temperature detector 40 also has the capability of measuring current flow through the liquid sample 28 passing between the first electrode 38 and the second electrode provided by the distal end 42. The distal end 42 therefore also contains a conductivity probe. Preferably also, the resistance temperature detector 40 is capable of detecting the temperature of the liquid sample 28.

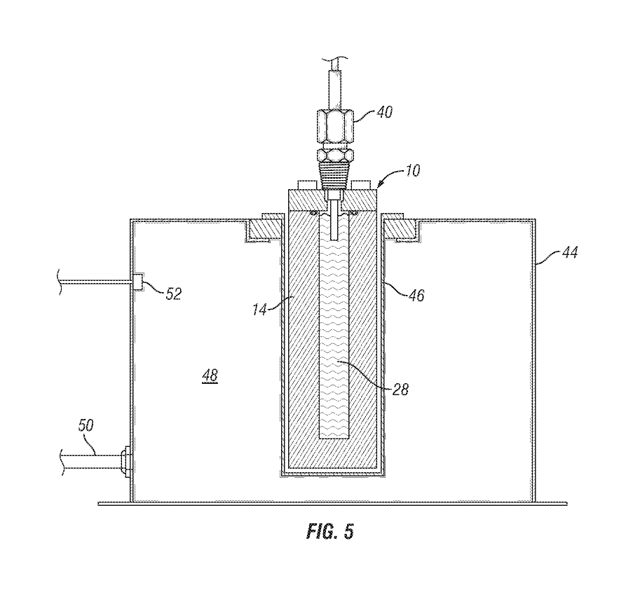

FIG. 5 illustrates the conductivity cell 12 now having been disposed within a heater block 44. The conductivity cell 10 is disposed within a conductivity cell sleeve 46 so that the testing vessel 14 is largely disposed within the interior chamber 48 of the heater block 44. A heater line 50 provides heated media to the interior chamber 48 to heat the liquid sample 28. A thermocouple 52 is associated with the heater block 44 to monitor the temperature at which the interior chamber 48 and conductivity cell 10 are maintained.

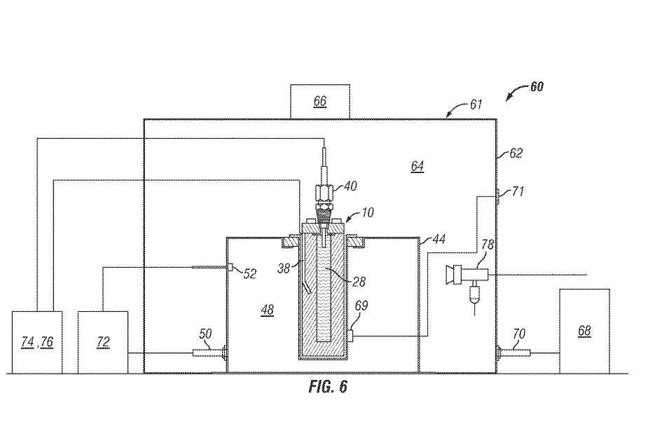

FIG. 6 illustrates a substantially complete conductivity meter 60 in accordance with the present invention wherein the heater block 44 is associated with a pressure control system 61 that is used to provide a pressure blanket of inert gas during testing. The heater block 44 is shown contained within a pressure vessel 62 which defines an interior pressure chamber 64. A relief valve 66 is preferably located on the top side of the pressure vessel 62 and preferably can be selectively opened and closed to permit depressurization of excess gas within the pressure chamber 64. A pressurized fluid source 68 is located proximate the pressure vessel 62 to supply a pressurized fluid to the pressure chamber 64 via conduit 70. The pressurized fluid is preferably an inert gas, such as ultrapure argon or nitrogen. If necessary, the pressurized fluid source 68 may include a fluid pump to generate desired pressures within the pressure chamber 64 as well as suitable valves to control the flow of pressurized fluid into the pressure chamber 64. A pressure transducer 69 is associated with the conductivity cell 10 and provides a signal indicative of measured pressure to pressure gauge 71, thereby enabling a user to control pressure within the pressure vessel 62 to achieve a desired pressure. The pressurized fluid source 68 is used to maintain the conductivity cell 10 and liquid sample 28 at a desired pressure. In addition, the pressurized fluid source 68 provides a pressure blanket which prevents or limits evaporation of the liquid sample 28 during testing. When an inert gas is used for the pressure blanket, an inert, oxygen-free environment is provided which prevents potential ignition of the sample during testing.

A heating controller 72 is operably interconnected with the thermocouple 52 and is operable to supply heated medium via conduit 50 into the interior chamber 48 of the heater block 44. The heating controller 72 maintains the temperature within the interior chamber 48 in accordance with temperature feedback provided by the thermocouple 52. In preferred embodiments, the heating controller 72 is a cascade feedback controller, of a type known in the art, which can provide optimized heating by attaining a desired temperature in a rapid manner.

The first and second electrodes 38, 40 are operably associated with a voltage power source 74 which are capable of supplying a voltage potential across the electrodes 38, 40 of the conductivity cell 10. The applied voltage might be an AC or a DC voltage. In preferred embodiments, the voltage power source 74 is capable of applying a constant DC voltage, such as 3V DC, for a predetermined length of time. In further preferred embodiments, the voltage power source 74 is capable of applying a variable range of DC voltages to the electrodes 38, 40. For example, the voltage power source 74 might be able to apply voltages within a range from 0-1000 V DC and be programmable to change between voltage levels in accordance with a predetermined scheme. A conductivity/resistance detector 76 is also operably associated with the conductivity probe of distal end 42 and is operable to detect the conductivity between the electrodes 38, 40 when a voltage is applied across them. The voltage source 74 and the conductivity/resistance detector 76 may be combined in a single device, as FIG. 6 illustrates. A suitable voltage source and conductivity/resistance detector for use as the components 74, 76 is a Keithley brand picoammeter, which is available commercially from Keithley Instruments, Inc. of Cleveland, Ohio. In particular embodiments, the voltage source 74 applies DC voltage in sequences of varying levels of voltage over short intervals of time (typically one second or less intervals). The application of pulsed DC field sequences, along short time intervals, is useful to overcome the effects of polarization, which could result in miscalculation of conductivity for a sample.

In accordance with particular embodiments, the conductivity meter 60 includes a cold gun 78 for rapidly cooling down the heater block 44 and conductivity cell 10 as a safety measure.

The conductivity meter 60 is useful to measure the conductivity of a sample 28 of non-aqueous liquid, such as crude oil. In operation, a sample 28 is placed within the liquid sample chamber 16 of the testing vessel 14 and cap 30 is affixed to the testing vessel 14. The resistance temperature detector 40 is inserted into the liquid sample 28 and the first electrode 38 is inserted into the electrode cavity 18. The conductivity cell 10 is then inserted into the conductivity cell sleeve 46 of the heater block 44 and the meter 60 further assembled in the manner depicted in FIG. 6. Next, the heating controller 72 is actuated to heat the heater block 44 and conductivity cell 10 to a predetermined temperature. The pressurized fluid source 68 adjusts the pressure within the pressure chamber 64 to a predetermined pressure level. Thereafter, a desired voltage level or levels is/are applied to the conductivity cell 10 by the voltage source 74 as conductivity is measured by the conductivity detector 76. The conductivity meter 60 allows measurement of conductivity over a range of temperatures and pressures as well as an over a range of applied voltages.

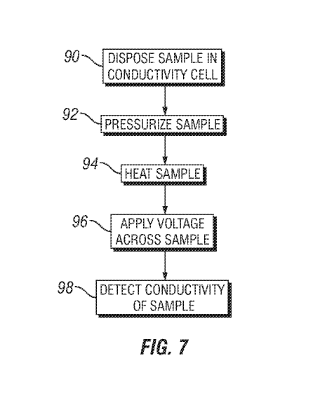

FIG. 7 depicts exemplary method steps for a method of measuring conductivity of a sample 28 of non-aqueous liquid in accordance with the present invention. In accordance with step 90 of the method, a sample 28 of non-aqueous liquid is disposed within a conductivity cell 10. In step 92, the conductivity cell 10 and sample 28 are pressurized to a predetermined pressure. Typically, this would be done by operation of the pressure control system 61 which provides a pressure blanket for the sample 28. In step 94, the conductivity cell 10 and sample 28 are then heated to a predetermined temperature by the heating controller 72. In step 96, a voltage is applied across the sample 28 by the voltage source 74. Conductivity of the sample 28 is then detected by the conductivity meter 60 as the voltage is applied in step 98.

Those of skill in the art will recognize that numerous modifications and changes may be made to the exemplary designs and embodiments described herein and that the invention is limited only by the claims that follow and any equivalents thereof.

1. A method for determining the conductivity of a sample of non-aqueous liquid, the method comprising the steps of:
disposing a sample of non-aqueous liquid within a conductivity cell;
applying a voltage across the sample of non-aqueous liquid; and
detecting conductivity of the sample of non-aqueous liquid as the voltage is applied across the sample.

2. The method of claim 1 wherein the step of applying a voltage further comprises applying a sequence of voltages of varying levels.

3. The method of claim 2 wherein the voltages within the sequence of voltages are each applied for a period of about one second.

4. The method of claim 1 further comprising the step of heating the sample of non-aqueous liquid to a desired temperature prior to applying the voltage across the sample.

5. The method of claim 1 further comprising the step of pressurizing the sample of non-aqueous liquid to a predetermined pressure prior to applying the voltage across the sample.

6. The method of claim 5 wherein the step of pressurizing the sample further comprises subjecting the sample of non-aqueous liquid to a pressure blanket of an inert gas.

7. The method of claim 1 wherein the step of disposing a sample of non-aqueous liquid within a conductivity cell further comprises:
placing the liquid sample within a testing vessel; and
immersing a resistance temperature detector within the sample.

8. The method of claim 3 wherein the step of heating the sample of non-aqueous liquid to a desired temperature further comprises:
disposing the conductivity cell within a heater block; and
controlling the temperature of the heater block with a heater controller.

9. A method for determining the conductivity of a sample of non-aqueous liquid, the method comprising the steps of:
disposing a sample of non-aqueous liquid within a conductivity cell;
pressurizing the sample of non-aqueous liquid to a predetermined pressure;
applying a voltage across the sample of non-aqueous liquid; and
detecting conductivity of the sample of non-aqueous liquid as the voltage is applied across the sample.

10. The method of claim 9 wherein the step of pressurizing the sample further comprises subjecting the sample of non-aqueous liquid to a pressure blanket of an inert gas.

11. The method of claim 9 further comprising the step of heating the sample of non-aqueous liquid to a desired temperature prior to applying the voltage across the sample.

12. The method of claim 9 wherein the step of disposing a sample of non-aqueous liquid within a conductivity cell further comprises:
placing the liquid sample within a testing vessel; and
immersing a resistance temperature detector within the sample.

13. The method of claim 11 wherein the step of heating the sample of non-aqueous liquid to a desired temperature further comprises:
disposing the conductivity cell within a heater block; and
controlling the temperature of the heater block with a heater controller.

14. A method for determining the conductivity of a sample of non-aqueous liquid, the method comprising the steps of:
disposing a sample of non-aqueous liquid within a conductivity cell;
heating the sample of non-aqueous liquid to a desired temperature;
applying a voltage across the sample of non-aqueous liquid; and
detecting conductivity of the sample of non-aqueous liquid as the voltage is applied across the sample.

15. The method of claim 14 further comprising the step of pressurizing the sample of non-aqueous liquid to a predetermined pressure prior to applying the voltage across the sample.

16. The method of claim 15 wherein the step of pressurizing the sample further comprises subjecting the sample of non-aqueous liquid to a pressure blanket of an inert gas.
